(19) United States
(12) Patent Application Publication
(10) Pub. No.: US 2012/0070898 A1
(43) Pub. Date: Mar. 22, 2012

(54) iCell

(76) Inventors: Carolyn Flora Anne Dean, Kihei, HI (US); Jeffrey Wallace Edwards, Bremen, GA (US)

(21) Appl. No.: 13/204,063

(22) Filed: Aug. 5, 2011

Related U.S. Application Data
(60) Provisional application No. 61/403,437, filed on Sep. 16, 2010.
(60) Provisional application No. 61/403,432, filed on Sep. 16, 2010.
(60) Provisional application No. 61/461,082, filed on Jan. 13, 2011.
(60) Provisional application No. 61/462,315, filed on Feb. 1, 2011.
(60) Provisional application No. 61/462,320, filed on Feb. 1, 2011.

(51) Int. Cl. C12N 5/00

(52) U.S. Cl. 435/410

(57) ABSTRACT

The iCell is a newly-created cell that is incubated and harvested using sprouted barley grain (or other gluten grains) along with clover seeds, pepper seeds, alfalfa water, and a live yeast sponge. An electrical current is used to stimulate the curing process to solidify the iCell in a contained field.

The iCell is a consistently-propagated cell that allows nutrition to be more completely utilized in the human body. This nutrition represented by Cellionose and enhanced amino acids allows for the creation of healthy cells throughout the body.

The iCell is identified as a new cellular organism whose properties have been encountered randomly throughout humanity's history. This patent outlines the production of consistently-propagated cells.

At a cellular level the iCell has far-reaching applications including water reclamation, water desalination, human hydration, and unique polypeptide bonding creating new amino acids. ICells have a direct correlation with the measurable potential for hydrogen. The iCell is responsible for providing information to affect the body's DNA via RNA.

### CROSS-REFERENCE TO RELATED APPLICATIONS

### Cellionose: U.S. 61/403,437

### Super Trihydrogen: U.S. 61/403,432

### Insulinose: U.S. 61/461,082

iBlock: U.S. 61/462,315

### Axeion Water: U.S. 61/462,320

### STATEMENT REGARDING FEDERALLY SPONSORED RESEARCH OR DEVELOPMENT

NA

### REFERENCE TO SEQUENCE LISTING, A TABLE, OR A COMPUTER PROGRAM LISTING COMPACT DISC APPENDIX

NA

### BACKGROUND OF THE INVENTION

The process to consistently create iCells is the focus of this patent.

The iCell invention pertains to the field of enhanced cellular replication.

The iCell is the original missing element that has been eliminated from the homo sapiens diet.

Facts about the iCell can be found in ancient history.

The original recipe for iCell is referenced in Leviticus and refers to “shew bread.” Shew bread was used only for sacrifices to God.

Manna is described in Deuteronomy as the perfect nutrition provided for the Hebrew people as they roamed the desert for 40 years. It was also called the “Staff of Life.” This historic event is still significant based on how humans view complex carbohydrates even today. The body digests and assimilates complex carbohydrates and they become a necessary nutritional building block for the body.

More recently, people have been rejecting bread as the staff of life. The iCell patent uniquely addresses the rejection of carbohydrates by our society.

A major stumbling block with the historical iCell is that the method was never consistently repeated. This process would occasionally result in the production of iCells. The presence of the iCell in Manna is what gave the bread that delectable, desired taste. And, nowadays, it is highly unlikely that iCells are inadvertently produced because few people make bread from scratch where they incubate the dough and keep a “bread starter” generation after generation.

The human body naturally craves any nutrition that it lacks. You crave tomatoes when you are low on lycopene. If you have iron-poor blood, you crave meat.

The natural way your body desires foods balances its nutrition. Since this unique cell, the iCell, has been mostly eliminated from our nutrition, the body doesn't even know what it's missing.

With this treasure missing, your body (not your mind) says you don't have the iCell so you have to keep eating more and more bread to try to attain it. Eating so much bread can lead to many health problems.

However, with the iCell, your body will naturally have the thing that allows it to be nurtured. Therefore, any other nutrition that you embrace will now be used differently.

As with grains, sugars of all kinds have almost been eliminated or blacklisted from our diet. And the way the body handles sugar is gauged to have a less than beneficial effect on the body with diagnoses of diabetes and hypoglycemia. However, if you completely eliminate all sugar, you can't process amino acids. You have to have energy to drive the life-giving metabolic processes in the body.

You are operating a combustion engine without the necessary motor oil. All the basic dietary building blocks that people eat are absolutely healthy for you in all levels and amounts if the iCell is included in your normal diet. Whatever you want to eat, you can when you take the iCell. Foods that the body can't utilize will be naturally eliminated without any reaction, or you will simply desire less of those foods.

Cell membranes are positively affected by the iCell. A cycle of production ensues within the cellular cytoplasm promoting healthy cells and the creation of new stabilized amino acids like trimethyllysine.

There is a relationship between the iCell and amino acids (lysine and trimethyllysine), lipids, phospholipids and picominerals that allows the cell's ion channels within the cell membrane to become more permeable to necessary nutrients.

The iCell process enhances the production of trimethyllysine, a necessary component in the transport and breakdown of lipids. Trimethyllysine is a component of histone proteins; a precursor of carnitine; and the coenzyme of fatty acid oxidation.

Carnitine is created from lysine and methionine making a quaternary ammonium compound. In living cells, it transports fatty acids from the cytosol into the mitochondria as lipids are being broken down to generate metabolic energy.

### BRIEF SUMMARY OF THE INVENTION

ICells occur in nature as do naturally-occurring yeast spores which are ambient and not specific to a particular environment and have an effect on other organisms. ICells have appeared inadvertently throughout man's existence but remained inconsistently identified until now. The essence of this patent is the procedure of how to consistently create and harvest iCells.

The iCell is a consistently-propagated cell that allows nutrition to be more completely utilized in the human body. The iCell is identified as a new cellular organism whose properties have been encountered throughout humanity's history. This patent outlines the production of consistently-propagated cells.

At a cellular level the iCell has far-reaching applications including water reclamation, water desalination, human hydration, and unique polypeptide bonding creating new amino acids. The iCell is responsible for providing information to affect the body's DNA via RNA. And iCells have a direct correlation with the measurable potential for hydrogen.

### BRIEF DESCRIPTION OF THE SEVERAL VIEWS OF THE DRAWING

NA

### DETAILED DESCRIPTION OF THE INVENTION

The iCell is a consistently-created cell that is incubated and harvested using sprouted barley grain (or other gluten grains) along with clover seeds, pepper seeds, alfalfa water, and a live yeast sponge. An electrical current is used to stimulate the curing process to solidify the iCell in a contained field.

iCell Procedures

### 5-Step Process for Making ICell

| | |
|---|---|
| Introduction: | p. 2 |
| Ingredients for 10 lb iBlocks | p. 2 |
| Procedure 1: Soaking the Grains | p. 3 |
| Procedure 2: Sprouting the Grains | p. 4 |
| Procedure 3: Making iCell Mash For The iBlock | p. 5 |
| Procedure 4: Preparing the iBlock Base | p. 7 |

### APPENDICES

| | |
|---|---|
| A-1a: | |
| Alfalfa Water (Soaking Water) | p. 9 |
| A-1b: | |
| Soaking and Sprouting Record | p. 9 |
| A-2a | |
| Shoot Juice | p. 9 |
| A-2b | |
| Berry Juice | p. 10 |
| A-2c | |
| Yeast Solution | p. 11 |
| A-2d | |
| Red Top Clover | p. 12 |
| A-2e | |
| Soaking Formula | p. 13 |
| Appendix for Succeeding Generations of iCell Production | p. 14 |

### Introduction:

This process creates iCells by incorporating grains in the stages of soaking, sprouting and curing into a block of clay forming a granite-like consistency that will be detailed below.

We can now isolate and purvey iCells which were previously only sporadically encountered.

Ingredients for 10 lb iBlocks

1. H₂O (to be replaced by Axeion water (patent pending))
2. Barley seeds (varieties including but not limited to Charles, Thoroughbred, Camas, Triticale)

### 3. Alfalfa hay

4. Red Top Clover seeds
5. Grains of Paradise peppercorns
6. Inoculant for Red Top Clover Seeds (carbon based)
7. Yeast—Brewer's yeast
8. White table sugar
9. Unbleached, unbromated white flour
10. Unsalted dehydrated instant potato flakes
11. Alfalfa water (Appendix A-1a) (used initially then change to Axeion water)

### 12. Berry Juice (Appendix A-2b)

13. Shoot juice (Appendix A-2a)

Materials:

1. Digital gram scale
2. Thermometer 32-150 F with stainless steel stem 6″ with protective cover
3. Digital pH meter
4. Digital timer

### 5. Measuring cup

6. Covered container (2 quart)
7. Growing trays (customized for this process)
8. Clear hot/cold liquid storage containers
9. Stainless steel (screened colanders)

### 10. Parchment

11. Porcelain ramekins
12. Spray bottles
13. Hypodermic syringe 10 cc (for spraying trays with yeast solution)
14. Incubator (110 volt table top incubator, internal volume approx. ¾ cubic foot: 0 F-120 F)
15. Transformer (110 volts in, 0-120 volts out)
16. 16-gauge insulated braided copper wire for leads
17. Copper tubing (copper tubes)
18. Specific gravity hydrometer
19. Digital multi meter (measuring ohms to 0.001)
20. Alligator clips
21. ¼″ OD flexible polyethylene tube approximately 24″
22. Straining cloth (layers of cheese cloth)
23. Mixing bowls (various sizes)
24. 6″ table top fan
25. Industrial paint mixing auger
26. Portable electric drill

### 27. Processor

### 28. Wisk

29. 7′ diameter clay saucer
30. Hand-held wire strainer

Observation Documentation:

### Sprouting Specifications Data Sheet (SSDS)

Procedure 1. Soaking the Grains

Ingredients:

1. H₂O (to be replaced by Axeion water)
2. Barley seeds
3. Soaking Water (Alfalfa Water to be replaced by Berry Juice and Axeion water) (Appendix A-1a)

### 4. Shoot Juice (Appendix A-2a)

### 5. Berry Juice (Appendix A-2b)

Materials:

1. 2-quart hard-plastic measuring cup

### 2. Digital Gram Scale

3. 6-Quart-Stainless Steel Screened Colander (medium fine)
4. Gallon Tupperware container
5. 1-quart Tupperware storage container with cover
6. Growing trays (specifications) 10″×20″
7. pH meter (electronic)
8. Thermometer 32-150 F with stainless steel stem 6″ with protective cover
9. 2, 1 Gallon Tupperware containers
10. Relative humidity gauge

### 11. Barometer

Procedure:

1. Measure 200 grams of three varieties of barley seeds for a total of 600 grams.

Place the seeds into a 6-quart, 18/10 stainless steel colander.

3. Rinse the seeds using non-chlorinated water. Gently hand-rub the seeds against the sides of the colander to remove any surface dirt.

4. Place the seeds into a gallon Tupperware container.

5. Use Alfalfa Water as the current Soaking Formula (Appendix A-1a) Maintain ⅜″ extra Alfalfa Water in the container. This procedure will produce Berry Juice and Axeion Water (which will become the new Soaking Formula replacing Alfalfa water in subsequent generations) referenced in the Moon Phase Chart (Appendix A-2e)

6. Place the gallon Tupperware container in the refrigerator for 8-10 hours at approximately 58° F.

7. Carefully document all aspects of the process using the Soaking and Sprouting Record (Appendix A-1b).

8. Remove from refrigerator. Pour the seeds in a colander and collect the unabsorbed Soaking Formula for later use. Rinse the seeds with H₂O (to be replaced by Axeion water) for 30 seconds agitating the seeds in the colander. Retain excess water that is drained out.

Procedure 2. Sprouting the Grains

Ingredients:

1. Soaked barley grains

### 2. Red Top Clover (Appendix A-2d)

### 3. Yeast Liquid (Appendix A-2c)

4. Grains of paradise peppercorns

### 5. Soaking Formula (Appendix A-1a)

Materials:

1. Growing trays
2. Spray bottle

### 3. Sprouting and Seed Chart

Procedures:

1. Place properly soaked seeds in a growing tray (from Procedure 1).

2. Spread the seeds evenly in the growing tray. Place 30 Grains of Paradise peppercorns in the middle of the tray (subsequent generations will use Grains of Paradise rhizomes).

3. Mix sprouted red top clover throughout the seed mixture.

4. Spray seeds with Soaking Formula.

5. Cover the tray for 12 hours at approximately 58 F, noting all variables.

6. Spray with Soaking Formula every 12 hours to keep seeds moist until fully germinated.

7. Every other day put fresh Yeast Solution in the refrigerator in ramekins. (Appendix A-2c). On those same days, also spray the trays with an ounce of yeast solution

8. Record percentage of seeds that are either sprouted, swollen or no change.

9. This process ends with the production of fully sprouted and/or swollen seeds which may be up to 14 days.

10. Rotate the trays daily for temperature consistency.

Procedure 3: Making iCell Mash for the iBlock

Ingredients:

1. Tray(s) of sprouted seeds (now called berries)

### 2. Yeast Solution (Appendix A-1a)

Materials:

### 1. Processor

2. Mixing bowls (3-4)
3. Quart jars (3)
4. pH meter (electronic)
5. 1 gallon pitcher (clear)
6. Stainless steel screened colander
7. Thermometer 32-150 F with stainless steel stem 6″ with protective cover

### 8. Shears

9. Measuring spoon

### 10. Spatula

11. Multi Meter for measuring ohms
12. Reusable ¼″ OD flexible polyethylene siphon tube approximately 24″

### Procedures:

Continuing from Procedure 2 (Sprouting the Grains) this process begins with fully sprouted/swollen berries and creates the iCell Mash for the iBlock.

### Day One

1. Preparing the mash.
a. Remove the trays from the refrigerator. If the berries are sprouted with green vertical growing shoots cut the green shoots from the berries at the base. Divide the berries/roots from the green shoots and place in two separate mixing bowls. If berries are not sprouted, proceed to Step 2.
b. If the berries have sprouted with a vigorous root mat, cut into 2″×2″ squares. Place two cups of the squares or swelled berries in processor and pulse for 30 seconds or until the berries are liquefied.
c. Transfer the processed berries from the processor into a large bowl. Continue this process until all trays have been processed.

2. a. Add to the processed berry mash:
2 oz Yeast Solution (Appendix A-2c)
8 oz Alfalfa Water (future generation use 8 oz Axeion water)
See Appendix A-2e for future generation addition of Sprout Juice and Berry Juice.
b. Mix well and put in an 85° F. incubator for 24 hours. Stir twice a day. After 24 hours the mixture will smell like sour mash.

### Day 2

3. Remove from incubator leaving uncovered at room 70° F. for 8 hours. Aerate the liquid mash.

### Day 3

4. Separate berry juice from organic matter. Use a fine screen steel colander to separate the mash from the liquid. Put a handful of mash through the colander, allowing the liquid to drain into a receptacle below. Once separated set aside in another bowl.

5. Transfer the liquid into glass jars and store in a refrigerator 40° F. labeled with date and germination record. This liquid is now called Berry Juice (Appendix A-2b). Patent Pending, Insulinose.

6. Remove 25% of the iCell Mash and label as Generation 1, and store in refrigerator. Transfer the remaining strained mash clumps into a sealed container and label this product, iCell Mash for iBlock.

7. If seeds have not sprouted and only swelled and expanded, extract liquid from mash as described above. However, this liquid must ‘settle’ allowing the heavy organic material to fall to the bottom of the container. The liquid above the organic material is extracted by siphoning off the liquid through a ¼″ OD flexible polyethylene tube approximately 24″ long and retained in a separate glass jar.

Procedure 4: Preparing the iBlock Base

Ingredients:

### 1. Berry Juice (Appendix A-2b)

2. iCell Mash

### 3. Clay

Materials:

1. 5 gallon plastic container.
2. Large 12-quart mixing bowl

### 3. Spatula

4. Curing tray
5. Straining cloth
6. 6″ table top fan
7. ¼″ OD copper tubes with 1″ 90 degree angle on both ends
8. Industrial paint mixing auger
9. Portable electric drill
10. Transformer (110 volts in, 0-120 volts out)
11.2 Copper leads
12. Alligator clips

Procedures:

1. Place the iCell Mash yield in a 5-gallon plastic container. Add one part clay to one part Mash and ¾ part H₂O (to be replaced by Axeion water). Mix well to a thick pudding consistency that does not adhere to the sides of the container. (Add more H₂O if necessary).

2. Mix to a dough-like consistency. Remove the mixture from the 5-gallon container and place in a curing tray lined with straining cloth, filling the tray to the edge of all sides.

3. Cut 2, ¼″ OD copper tubes the length of the curing tray, bend tubes 90 degrees, at the 2 inch make on each end. Embed one of the copper tubes along one axis of the tray length into the mixture. Place the 2^{nd} copper tube on the opposite side of the tray. Expose 1 inch of the copper tube above the surface of the clay/mash. Place a steel stem digital thermometer in the mixture close to (but not touching) one of the copper tubes to monitor the mixture temperature.

4. Connect the iBlock copper tubes to a transformer by applying one lead from the transformer output to the copper tube. Take the second lead from the transformer and attach to the multi meter. Apply lead from the multi meter and attach to the second copper tube on the opposite end. Adjust multi meter to 2 decimal points to gauge amperage.

5. Every 15 minutes note temperature. Increase transformer voltage to a maximum of 0.75 AMPS. Note: Do not exceed 0.75 amps as the temperature of the iBlock will exceed 110° F. Ideal temperature is 85° F. internally.

6. Provide air circulation over the surface of the iBlock using a fan.

7. After 8 hours, remove the leads as the iBlock begins to separate from the sides of the curing tray. Remove the iBlock and place on drying rack. The straining cloth will adhere to the mixture. Reconnect the leads to the AMP. A temperature alarm should be set not to exceed 85° F. Circulate air on the surface of the iBlock.

8. Gradually increase the voltage to maintain 0.75 AMPS until voltage transformer is maxed out at 120 volts, the amperage will continue to decrease. The process is complete when at full voltage the amperage is reduced to 0.001. Remove wire leads, remove straining cloth. Add Ohm meter to the copper tubes. Allow 5 minutes for the ohmerage to stabilize, record. Elevate iBlock on drying rack. Record the ohmerage and weight daily for 14 days.

### APPENDICES

A-1a: Alfalfa Water (Becomes Soaking Mixture—See A-2e)

1) Fill a 5-gallon pail with clean dried alfalfa hay.

2) Fill pail ¾ full with H₂O.

3) Soak for 2 days in the sun uncovered, stirring occasionally.

4) Soak for 2 days out of the sun. Record pH.

5) Strain organic matter. Place liquid in containers and refrigerate.

6) Fill a 10-16 oz. spray bottle with alfalfa water.

7) Spray sprouts, 20 hours after the initial soaking.

8) Check solution's pungency. Strong smell indicates spoilage. Discard.

9) As soon as Axeion water is achieved, alfalfa water will no longer be required.

And will become Soaking Mixture.

A-1b: Seeding and Sprouting Record

| | | | | | | | | | | |
|---|---|---|---|---|---|---|---|---|---|---|
| | | | | | | | New | | | |
| | Moon | Germination/ | | Room | | | Yeast | H₂O | BJ | |
| Date | Phase | # day | Weight | Temp | Bara | Humid | Y/N | pH | pH | Comments |

A-2a: Making Shoot Juice

### Ingredients:

1. 2-4 Berry mats
2. Previous generation Shoot juice (A-2a)
3. Previous generation Berry juice (A-2b)
4. Axeion water

### Materials:

### 1. Shears

2. 2 glass mixing bowls (1-2 quarts)

### 3. Processer

4. 2, 1-quart jars

### Procedure:

1. Prepare small mats of fully matured berries.
2. Using shears remove the green shoot from the berry at the base of the shoot. Divide the berries from the green shoots and place in two separate mixing bowls.
3. Transfer the green shoots into a processor, and add a 50/50 blend of Berry Juice and Axeion water.
4. Add 1-2 oz of previous generation Shoot Juice.
5. Liquify in processor to a foamy consistency.
6. Let the Shoot Juice settle for 24 hours at 70° F.
7. Strain the liquid from the pulp. Bottle, date and identify the Shoot Juice by its generation and store at 40° F.
8. Retain the pulp in a recycle container.
9. Retain the berries to make an iBlock.

A-2b: Making Berry Juice

### Ingredients:

1. Tray(s) of berries

### 2. Yeast Solution (Appendix A-2c)

3. iCell Mash

### Materials:

### 1. Processor

2. Mixing bowls (3-4)
3. Quart jars (3)
4. pH meter
5. 1 gallon pitcher (clear).
6. Stainless steel strainer
7. Thermometer 32-150 F with stainless steel stem 6″ with protective cover
7. Transformer (110 volts in, 0-120 volts out)
8. 2 Copper leads
9. ¼ inch OD Type J copper tube
9. Alligator clips

### 10. Shears

11. Measuring spoon

### 12. Spatula

### Procedures:

The iCell Mash is a by-product called Berry Juice.

### Day One

1). Preparing the mash.
a. Remove the trays from the refrigerator. If the berries are sprouted with green vertical growing shoots cut the green shoots from the berries at the base. Divide the berries/roots from the green shoots and place in two separate mixing bowls.
If berries are not sprouted, proceed to Step 2.
b. If the berries have sprouted with a vigorous root mat, cut into 2″×2″ squares. Place two cups of the squares or swelled berries in processor and pulse for 30 seconds or until the berries are liquefied.
c. Transfer the processed berries from the processor into a large bowl. Continue this process until all trays have been processed.
   2). a. Add to the processed berry mash:
2 oz Yeast solution
8 oz Alfala Water (future generation use 8 oz Axeion water)
See Appendix A-3a for future generation addition of Sprout Juice and Berry Juice.
b. Mix well and put in an 85° F. incubator for 24 hours. Stir twice a day. After 24 hours the mixture will smell like sour mash.

### Day 2

4. Remove from incubator leaving uncovered at 70° F. for 8 hours. Aerate the liquid mash.

### Day 3

5. Separate berry juice from organic matter. Use a fine screen steel colander to separate the mash from the liquid. Strain a handful of mash in the colander, allowing the liquid to drain into a receptacle below. Once separated set aside in another bowl.
6. Transfer the liquid into glass jars and store in a refrigerator 40° F. labeled with date and germination record. This liquid is now called Berry Juice (Appendix A-2b).

A-2c—Process—Making Yeast Starter (Sponge) to Create Yeast Solution

### Ingredients:

### 1. H₂O

2. Yeast Solution (in future generations)

### 3. Brewer's Yeast

4. Potato flakes
5. White sugar
6. Unbleached unbromated white flour

### Materials:

1. Thermometer 32-150° F. with stainless steel stem 6″ with protective cover
2. Covered Pyrex dish
3. Measuring spoon

### 4. Wisk

5. Six Porcelain ramekins

### Procedure:

1. Mix at 70° F., 45 grams of Brewer's Yeast and 4 teaspoons of white table sugar and 8 oz of (less than) 105° F. H₂O in a covered glass dish. Wait 15 minutes.
2. As the yeast proofs (bubbles) add 4 teaspoons of plain potato flakes and 4 teaspoons of unbleached, unbromated white flour to the yeast sponge.
3. Evenly distribute half the yeast sponge into several ramekins. Reserve the other half as the Yeast Sponge. The liquid used in the ramekins is now called Yeast Solution.
4. Place the ramekin of Yeast Solution uncovered, into the sprouting area in close proximity to the sprouting seeds.
5. Rotate a new ramekin of Yeast Solution in sprouting area every 2 days.
6. Thin Yeast Solution with up to 8 oz of water as needed to spray berries.
7. Using the Yeast Sponge process, dilute by half to create Yeast Solution as needed always keeping half back as the Yeast Sponge. Each time you use half the Yeast Sponge add the Brewers's yeast, sugar, potato flakes and up to 8 oz of water to feed and replenish the Sponge.

A-2d—Process—Seeding and Sprouting Red Top Clover (RTC)

### Ingredients:

1. Red Top Clover seeds
2. Berry juice (Appendix A-2b)
3. Sprout juice (Appendix A-2a)
4. Inoculant for Red Top Clover Seeds (carbon based)

### Materials:

1. 8 oz glass container
2. Measuring spoons
3. 7″ diameter clay saucer

### 4. Parchment

5. Hand-held wire strainer (or food mill)
6. Spray bottle

### Procedure:

1. Pour 2 oz of Axeion water in a clay saucer and let sit for 24 hrs.
2. Place 2 TBSP of RTC seeds in an 8 oz glass container. Soak seeds in H₂O (to be replaced by Axeion water) for 24 hours at 70° F.
3. Add ⅛^{th} tsp of Inoculant to the red top clover and mix well.
4. Distribute a single layer of clover seeds onto the clay saucer. Cover the dish with a single sheet of parchment, and place in an incubator at 85° F. for 3 days.
5. Check the moisture level of the seeds every 12 hrs. Increase moisture as necessary.

A-2e: Soaking Formula—Moon Phase Chart:

Mix Berry Juice (Appendix A-2b) with Axeion Water in the following proportions following the Moon Phase to create the Soaking Formula.

### Moon Phase Chart:

New moon: 100% Berry Juice

### Quarter Moon: 50:50 Berry Juice: Axeion Water

### Full Moon: 75% BJ: Axeion

Last Quarter to New Moon: 100% Axeion water.

A-3a: Appendix for Succeeding Generations of iCell Production

These instructions will be used in succeeding generations of iCell production.

Procedure 3: Making iCell Mash for the iBlock Ingredients

Include under Ingredients:
1. Previous generation Berry juice (Appendix A-2b)
2. Previous generation Sprout juice (Appendix A-2a)
3. Previous generation iCell Mash from iBlock
Include under Procedures #2a.
2 oz Sprout juice (Appendix A-2a)
8 oz Berry juice (Appendix A-2b)

Procedure 4: Preparing IBlock Base

Include under Ingredients:
1. Previous generation iCell Mash
2. Clay (use previous generation clay if available)

A-1a—Process: Making Alfalfa Water

Include under Ingredients:
Include a 1-pound iBlock to the mixture. New mixture will be known as Axeion Water.

A-2b—Process—Making Berry Juice

Include under Ingredients:
Previous generation Berry juice (Appendix A-2b)
Previous generation Sprout juice (Appendix A-2a)
Previous generation iCell Mash from iBlock

### DRAWINGS

### When Necessary

NA

### DRAWING REQUIREMENTS SECTION

NA

### SEQUENCE LISTING

### When Necessary

NA

1. The iCell shall change the manner in which the human body assimilates nutrition.

2. The iCell shall modify the body's means of replicating healthy cells.

3. The iCell shall alter the way hydrogen is processed through photosynthesis.

4. The isolated iCell has unlimited potential for enhancement of living matter.

5. The iCell is the key building block of Super Trihydrogen (#U.S. 61/403,432), which creates the new stable chemical C122 H197 098 (Insulinose) (#U.S. 61/461,082).

6. The iCell is key to enhancing amino acids within the human lipid system and creating stable new amino acids.
